(19) United States
(12) Patent Application Publication
(10) Pub. No.: US 2012/0220827 A1
(43) Pub. Date: Aug. 30, 2012

(54) SHAPE MEMORY ARTHROSCOPIC CAMERA

(75) Inventors: Timothy Sargeant, Guilford, CT (US); Arpan Desai, Hamden, CT (US); Atu Agawu, Princeton, NJ (US)

(73) Assignee: Tyco Healthcare Group LP

(21) Appl. No.: 13/367,593

(22) Filed: Feb. 7, 2012

Related U.S. Application Data
(60) Provisional application No. 61/447,238, filed on Feb. 28, 2011.

(51) Int. Cl. A61B 1/04

(52) U.S. Cl. 600/109

(57) ABSTRACT

A surgical instrument includes an elongated tubular member having a proximal end and a distal end. At least a portion of the elongated tubular member is resiliently deformable from an at-rest configuration. The elongated tubular member includes a camera assembly disposed therein. An outer shaft defining a lumen extending therethrough is positioned annularly about the elongated tubular member. At least one of the outer shaft and the elongated tubular member is moveable with respect to the other between a substantially overlapping position wherein the elongated tubular member is deformed to conform to the shape of the outer shaft, and a displaced position wherein at least a portion of the elongated tubular member extends distally from a distal end of the outer shaft. In the displaced position, the portion of the elongated tubular member extending from the outer shaft returns, or reforms back to the at-rest configuration.

### CROSS REFERENCE TO RELATED APPLICATION

The present application claims the benefit of and priority to U.S. Provisional Application Ser. No. 61/447,238, filed on Feb. 28, 2011, the entire contents of which are incorporated herein by reference.

### BACKGROUND

1. Technical Field

The present disclosure relates to a surgical instrument, and more particularly, to an arthroscopic camera for providing better visualization within the joint space.

2. Background of Related Art

Endoscopic surgical procedures are minimally invasive procedures in which operations are carried out within the body by using elongated instruments inserted through small entrance openings in the body. Arthroscopic surgical procedures, a subset of endoscopic surgical procedures, are minimally invasive procedures carried out within the interior of a joint, i.e., the joint space.

Minimally invasive procedures, e.g., arthroscopic procedures, are desirable in that they allow for quicker recovery time and shorter hospital stays as compared to open surgical procedures. Minimally invasive procedures also leave minimal scarring (both internally and externally) and reduce patient discomfort during the recovery period. However, because the interior dimensions of the entrance openings into the body are necessarily small, only elongated, small diametered instrumentation may be used to access the internal joint space.

During an arthroscopic procedure, for example, an arthroscope, an elongated tubular instrument that allows a surgeon to illuminate and view the joint space, is inserted into the interior of the joint through a small incision. As can be appreciated, the maneuverability of the arthroscope is limited by the dimensions of the joint space as well as by the dimensions of the entrance opening into the body. Thus, due to the spatial constraints of arthroscopic procedures, the ability to maneuver the arthroscope within the interior of the joint to illuminate and view the joint space is limited.

### SUMMARY

In accordance with one embodiment of the present disclosure, a surgical instrument is provided. The surgical instrument includes an elongated tubular member having a proximal end and a distal end. A portion of the elongated tubular member (or the entire elongated tubular member) is resiliently deformable from an at-rest configuration to, for example, a curved configuration. An outer shaft defining a lumen extending therethrough is positioned annularly about the elongated tubular member. The outer shaft and/or the elongated tubular member are moveable with respect to each other between a substantially overlapping position and a displaced position. In the substantially overlapping position, the elongated tubular member is positioned within the outer shaft and is deformed to conform to the shape of the outer shaft. In the displaced position, a portion of the elongated tubular member extends distally from a distal end of the outer shaft such that the portion of the elongated tubular member extending from the outer shaft returns back to the at-rest configuration, e.g., the curved configuration.

In one embodiment, the elongated tubular member is formed from a shape memory material. More specifically, the elongated tubular member may be formed from nitinol.

In another embodiment, the outer shaft defines a substantially linear configuration. Further, the outer shaft may be formed from a rigid or a semi-rigid material. Accordingly, the elongated tubular member may define a substantially linear configuration when positioned within the rigid outer shaft in the substantially overlapping position.

In still another embodiment, the distal end of the elongated tubular member is curved off a longitudinal axis of the elongated tubular member by about zero degrees to about 180 degrees with respect to the longitudinal axis when the elongated tubular member is in the at-rest (curved) configuration.

In yet another embodiment, a camera assembly is disposed within the elongated tubular member. The camera assembly is configured to transmit an image from the distal end of the elongated tubular member to a remotely positioned video display for displaying the image as a video image. The image may be an image of an area extending outwardly from and in the direction of the distal end of the elongated tubular member.

In still yet another embodiment, the elongated tubular member is rotatable with respect to the outer shaft about a longitudinal axis of the outer shaft.

In accordance with another embodiment of the present disclosure, a surgical camera is provided. The surgical camera includes an elongated tubular member having a proximal end and a distal end. The elongated tubular member is resiliently deformable from an at-rest curved configuration to a substantially linear configuration. An image sensing and transmitting assembly is disposed within the elongated tubular member. The image sensing and transmitting assembly is configured to receive an optical image at the distal end of the elongated tubular and to transmit the image through the elongated tubular member, ultimately communicating the image to a remotely positioned video display for displaying the image as a video image. An outer shaft is disposed annularly about the elongated tubular member. The outer shaft and/or the elongated tubular member are longitudinally slidable with respect to each other between a substantially overlapping position and a displaced position. In the substantially overlapping position, the elongated tubular member is deformed by the outer shaft to the substantially linear configuration. In the displaced position, the elongated tubular member is reformed to the at-rest or curved configuration.

In one embodiment, the image is an image of an area extending distally along a longitudinal axis of the outer shaft when the elongated tubular member is in the substantially linear configuration. When the elongated tubular member is reformed back to the curved configuration, the image is an image of an area off the longitudinal axis of the elongated tubular member, extending from and in the direction of the distal end of the curved elongated tubular member.

Similar to the previous embodiment, the distal end of the elongated tubular member may be curved by about zero degrees to about 180 degrees when the elongated tubular member is in the curved configuration. Further, the elongated tubular member may be rotatable with respect to the outer shaft about a longitudinal axis of the outer shaft.

### BRIEF DESCRIPTION OF THE DRAWINGS

Various embodiments of the subject instrument are described herein with reference to the drawings wherein:

FIG. 1 is a side view of an arthroscopic camera in accordance with the present disclosure, shown disposed in a substantially overlapping, or linear configuration;

FIG. 2 is a side view of the arthroscopic camera of FIG. 1, where the arthroscopic camera is being transitioned from the substantially overlapping configuration to a displaced, or curved configuration;

FIG. 3 is a side view of the arthroscopic camera of FIG. 1, shown disposed in the displaced configuration;

FIG. 4 is a side view of the arthroscopic camera of FIG. 3, where an elongated tubular member of the arthroscopic camera has been rotated with respect to an outer shaft of the arthroscopic camera about a longitudinal axis thereof;

FIG. 5 is a side, cross-sectional view of the arthroscopic camera of FIG. 1, shown inserted through an opening in tissue in the substantially overlapping configuration; and

FIG. 6 is a side, cross-sectional view of the arthroscopic camera of FIG. 1, shown inserted through an opening in tissue in the displaced configuration.

### DETAILED DESCRIPTION

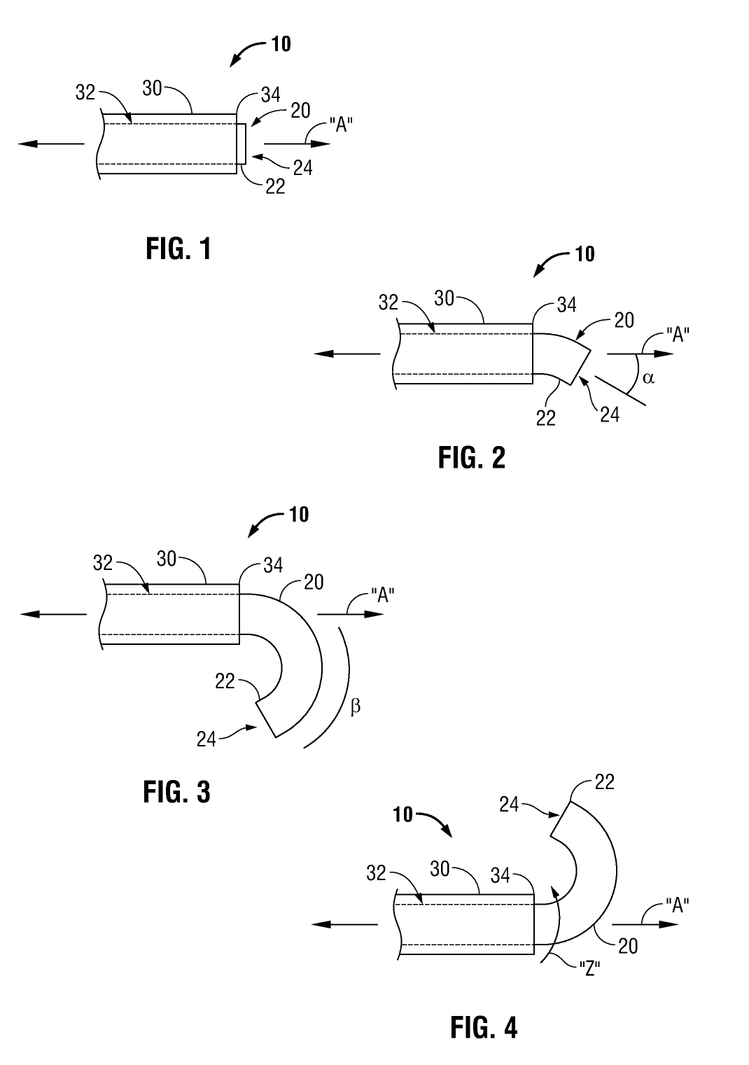

Referring now to FIG. 1, a surgical instrument for use in endoscopic and, more particularly, in arthroscopic surgical procedures is shown identified by reference numeral 10. Surgical instrument 10 generally includes an elongated tubular member 20 and an outer shaft 30 disposed annularly about elongated tubular member 20. Elongated tubular member 20 is slidable with respect to outer shaft 30 along a longitudinal axis “A.” More specifically, elongated tubular member 20 is translatable with respect to outer shaft 30 between a substantially overlapping position (FIG. 1) and a displaced position (FIG. 3). In the substantially overlapping position, elongated tubular member 20 is positioned within a lumen 32 extending through outer shaft 30. In the displaced position, elongated tubular member 20 extends distally from a distal end 34 of outer shaft 30.

Elongated tubular member 20 is resiliently deformable such that, when elongated tubular member 20 is positioned within outer shaft 30, i.e., when elongated tubular member 20 is in the substantially overlapping position, elongated tubular member 20 is conformed, or deformed to the shape of outer shaft 30. When elongated tubular member 20 is translated to the displaced position (FIG. 3), i.e., when elongated tubular member 20 is extended from outer shaft 30, the portion of elongated tubular member 20 extending distally from distal end 34 of outer shaft 30 is returned back to an at-rest position, or configuration. In other words, elongated tubular member 20 is deformable from its at-rest shape, but “remembers” the at-rest shape such that elongated tubular member 20 returns to the at-rest shape in the absence of a deforming force acting on elongated tubular member 20.

Elongated tubular member 20 includes a lumen 24 extending therethrough and is configured for housing the internal components of surgical instrument 10. Elongated tubular member 20 further includes an operative distal end 22. Elongated tubular member 20 may be configured, for example, as a surgical camera, e.g., an arthroscope, wherein image sensing and transmitting components (not shown) are disposed within lumen 24 of elongated tubular member 20. The image sensing and transmitting components receive and transmit an image of the area extending distally from operative distal end 22, i.e., the area directly in front of operative distal end 22, to a remotely positioned video display (not shown) for displaying the image as a video image. Alternatively, operative distal end 22 of elongated tubular member 20 may be configured to deliver fluids to a specific area within the body, or to direct light, thermal, and/or electrical energy to a specific area within the body. Put more generally, operative distal end 22 of elongated tubular member 20 may be configured for any suitable purpose wherein the specific orientation of operative distal end 22 determines, for example, the field of view or the direction of application of surgical instrument 10. Thus, as can be appreciated, elongated tubular member 20 may be transitioned between the substantially overlapping position and the displaced position to orient operative distal end 22 in a desired direction for viewing and/or treating a specific area within the body.

As mentioned above, elongated tubular member 20 is resiliently deformable from its at-rest shape. Elongated tubular member 20 may be made from any resiliently deformable material. In particular, elongated tubular member 20 may be made from a super elastic or shape memory material, such as nitinol, or any other suitable material that is flexible to permit repeated deforming, resilient to consistently return back to the at-rest shape, and strong, or rigid enough to maintain the at-rest shape. Potential materials for this application require high elasticity or super elasticity, capable of greater than 3% elastic strain and preferably capable of greater than 4% elastic strain. Optimal elastic strains are greater than 8%. Suitable materials in addition to shape memory materials, such as Nitinol, include rubbers and some plastics. It is also envisioned that elongated tubular member 20 be formed from, or coated with a bio-compatible material to reduce the incidence of adverse reaction by a patient upon contact with the patient's tissue.

Elongated tubular member 20 may be configured to define a “J-shaped” curved configuration when in the at-rest, or displaced position, i.e., in the absence of a deforming force acting thereon. More specifically, in the at-rest position, operative distal end 22 of elongated tubular member 20 may be curved by about 180 degrees of a longitudinal axis “A” thereof, such that operative distal end 22 is directed in a substantially opposite, or proximal direction relative to outer shaft 30. Alternatively, elongated tubular member 20 may be configured to define other curved configurations, e.g., an “L-shaped” curve or a snaked configuration including multiple curves. The specific at-rest shape of elongated tubular member 20 may be configured according to the surgical procedure to be performed and/or the internal body cavity dimensions wherein surgical instrument 10 is to be inserted.

Outer shaft 30 is an elongated hollow shaft 30 including a lumen 32 extending therethrough. As mentioned above, outer shaft 30 is disposed annularly about elongated tubular member 20, i.e., elongated tubular member 20 is positioned within lumen 32 of outer shaft 30, and is translatable with respect to elongated tubular member 20 about a longitudinal axis “A.” Outer shaft 30 may be formed from a rigid, or semi-rigid bio-compatible material. It is envisioned that outer shaft 30 be sufficiently rigid and have sufficient strength to retain its shape and to deform elongated tubular member 20 when elongated tubular member 20 is disposed within lumen 32 of outer shaft 30 (the substantially overlapping position). Outer shaft 30 may define a substantially linear configuration, as shown in FIGS. 1-6, however it is also envisioned that outer shaft 30 define a curved configuration. Further, outer shaft 30 may be configured and dimensioned for insertion through an incision in tissue to and for positioning within an internal body cavity, e.g., the joint space.

FIG. 2 shows elongated tubular member 20 translating, or sliding, with respect to outer shaft 30 from the substantially overlapping position of FIG. 1 to the displaced position of FIG. 3. Accordingly, as elongated tubular member 20 extends distally from outer shaft 30, the portion of elongated tubular member 20 extending from outer shaft 30 is no longer constrained by the dimensions of outer shaft 30 and, thus, is resiliently returned back to the at-rest, or “remembered” shape. In the position shown in FIG. 2, distal end 22 of elongated tubular member 20 is deflected, or curved off the longitudinal axis “A” at an angle “α.”

As shown in FIG. 3, elongated tubular member 20 has been translated to the displaced position wherein elongated tubular member 20 extends further from distal end 34 of outer shaft 30 and is thus returned further to the at-rest curved configuration. More specifically, the portion of elongated tubular member 20 extending from outer shaft 30 defines a “J-shaped” or 180 degree curved configuration such that operative distal end 22 is directed in a substantially proximal direction. This “J-shaped” configuration corresponds to the at-rest position of elongated tubular member 20. In this at-rest, or displaced position, distal end 22 of elongated tubular member is curved off the longitudinal axis “A” at an angle “β.” As can be appreciated, translating elongated tubular member 20 with respect to outer shaft 30 between the substantially overlapping position (FIG. 1) and the displaced position (FIG. 3) angles distal end 22 of elongated tubular member 20 with respect to longitudinal axis “A” from about zero degrees (FIG. 1, the substantially overlapping position), through angle “α” (FIG. 2, an intermediate position) and, ultimately, to angle “β” (FIG. 3, the displaced position).

A control member, e.g., a switch or a trigger (not shown), may be operably coupled to a proximal end of surgical instrument 10 for translating elongated tubular member 20 with respect to outer shaft 30 between the substantially overlapping position and the displaced position. The control member (not shown) may electrically or mechanically control the translation of elongated tubular member 20. Alternatively, translation of elongated tubular member 20 with respect to outer shaft 30 may be manually controlled.

A locking mechanism (not shown) may be provided to fix the position of elongated tubular member 20 with respect to outer shaft 30. The locking mechanism (not shown) may be controlled by a control member (not shown) or may be manually controlled. When the locking mechanism (not shown) is engaged, i.e., in a locked position, the position of elongated tubular member 20 may be fixed. More particularly, locking the locking mechanism may be used to fix elongated tubular member 20 in the substantially overlapping position (FIG. 1), wherein elongated tubular member 20 is deformed to the shape of and positioned within outer shaft 30, the displaced position (FIG. 3), wherein elongated tubular member 20 is resiliently returned back to the at-rest shape, or any intermediate position therebetween, e.g., the position of FIG. 2. Accordingly, distal end 22 of elongated tubular member 20 may be deflected, or repositioned, e.g., by translating elongated tubular member 20 with respect to outer shaft 30, and fixed with respect to longitudinal axis “A” between about zero degrees and angle “β,” e.g., about 180 degrees.

As shown in FIG. 4, elongated tubular member 20 may also be rotated with respect to outer shaft 30 about longitudinal axis “A,” as shown by arrow “Z.” A control member (not shown) may control rotation of elongated tubular member 20, or, alternatively, elongated tubular member 20 may be manually rotated with respect to outer shaft 30. As can be appreciated, the combination of deflecting distal end 22 of elongated tubular member 20 off longitudinal axis “A,” i.e., moving elongated tubular member 20 between the substantially overlapping and displaced positions, and rotating elongated tubular member 20 with respect to longitudinal axis “A,” allows for 360 degree positioning of distal end 22 of elongated tubular member 20 with respect to outer shaft 30. Further, rotating elongated tubular member 20, as opposed to rotating the entire surgical instrument 10, maintains an external surface of surgical instrument 10, e.g., outer shaft 30, in a fixed relationship with respect to tissue when inserted therethrough, thus minimizing the potential for tissue damage during positioning of distal end 22 of elongated tubular member 20.

As mentioned above, elongated tubular member 20 may house a camera assembly (not explicitly shown) therein. In other words, surgical instrument 10 may be configured as an arthroscopic camera 10. In such embodiments, a light source (not explicitly shown) is positioned within lumen 24 of elongated tubular member 20 at distal end 22 thereof. The light source (not shown) is configured to emit light from distal end 22 of elongated tubular member 20, i.e., to illuminate the field of view of arthroscope 10. The light source (not shown) may be an illuminated tip of a fiber optic bundle, a light emitting diode (LED), or any other suitable light emitting mechanism.

Image sensing and transmitting components (not shown), e.g., a camera, the fiber optic bundle, or other components, are positioned within elongated tubular member 20 and extend proximally through lumen 24 of elongated tubular member 20. The image sensing component (not shown) is positioned at distal end 22 of elongated tubular member 20 and is configured to receive an optical image, e.g., an image of the illuminated surgical site. The transmitting component (not shown) is configured to transmit the image through elongated tubular member 20, e.g., within the fiber optic bundle, to a remote, external video display monitor (not shown). The transmitting component may be coupled, at a proximal end of arthroscope 10, to an antenna (not shown) for wirelessly transmitting the image to the video display (not shown), or, alternatively, to a cable (not shown) for wired transmission of the image to the video display (not shown). Thus, in either configuration, the video display (not shown) may provide a video image of the surgical site, e.g., the joint space, as viewed from and in the direction of distal end 22 of elongated tubular member 20.

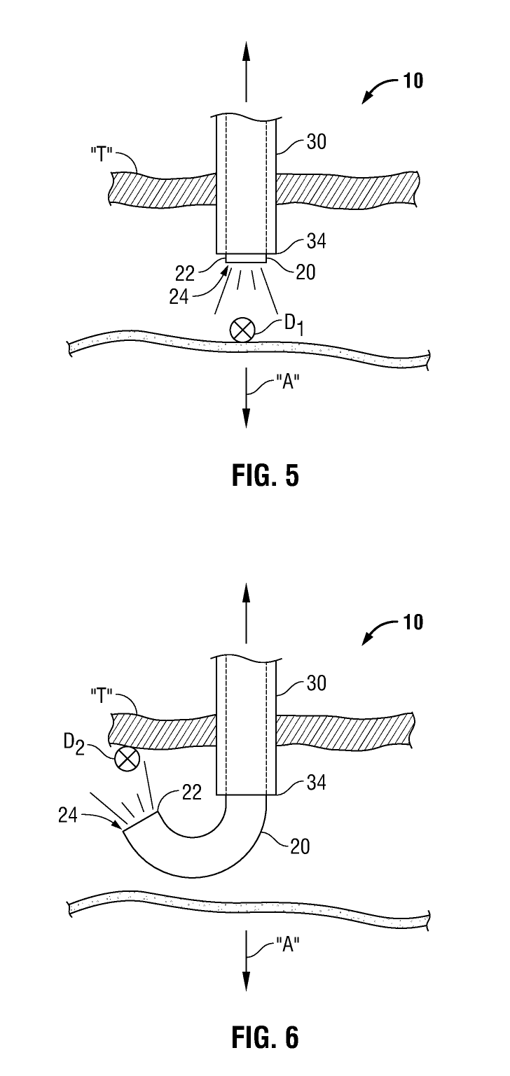

The operation of arthroscopic camera 10 will now be discussed with reference to FIGS. 5 and 6. Although the operation of surgical instrument 10 is described hereinbelow specifically as relating to arthroscopic camera 10, it is envisioned that other embodiments of surgical instrument 10, e.g., where surgical instrument 10 is a fluid dispenser or heat applicator, operate in a similar fashion.

Initially, as shown in FIG. 5, arthroscope 10 is moved in the substantially overlapping position wherein elongated tubular member 20 is positioned within lumen 32 of outer shaft 30. As such, elongated tubular member 20 is conformed to the dimensions, or shape of outer shaft 30, which, as shown in FIG. 5 is substantially linear. Thus, in the substantially overlapping position, arthroscope 10 defines a generally linear configuration. In this linear, or substantially overlapping position, arthroscope 10 may be inserted through an incision, or opening in tissue “T” and may be positioned within an internal surgical site, e.g., the joint space.

As can be appreciated, with arthroscope 10 inserted through an opening in tissue “T” in the substantially overlapping position, as shown in FIG. 5, the viewing direction, i.e., the field of view, of arthroscope 10 is the area extending distally along longitudinal axis “A.” More particularly, the viewing direction of arthroscope 10 is defined distally from and in the direction of distal end 22 of elongated tubular member 20. Thus, since distal end 22 of elongated tubular member 20 is aligned with longitudinal axis “A” in the substantially overlapping position, the viewing direction of arthroscope 10 is along longitudinal axis “A.” Accordingly, in this position, arthroscope 10 may be used to view a joint defect “D₁” (or other condition) positioned along, or in the area of longitudinal axis “A.”

Referring now to FIG. 6, in order to view a defect or other condition that is positioned offset from longitudinal axis “A,” e.g., defect “D₂,” elongated tubular member 20 may be moved from the substantially overlapping position within outer shaft 30 to the displaced position wherein elongated tubular member 20 extends distally from outer shaft 30.

As mentioned above, the control member (not shown) may be used to translate elongated tubular member 20 with respect to outer shaft 30 or, alternatively, elongated tubular member 20 may be manually translated with respect to outer shaft 30. As can be appreciated, as elongated tubular member 20 is extended from outer shaft 30, outer shaft 30 remains in a fixed position with respect to tissue “T,” reducing the likelihood of inadvertent tissue damage during manipulation of arthroscope 10 within the joint space.

As elongated tubular member 20 is extended distally from outer shaft 30 toward the displaced position, the portion of elongated tubular member 20 extending from distal end 34 of outer shaft 30 is resiliently returned back to the at-rest, or curved configuration, or shape. Thus, as elongated tubular member 20 is returned to the at-rest shape, distal end 22 of elongated tubular member 20 is angled off longitudinal axis “A” from about zero degrees (the substantially overlapping position) to the curved position, e.g., to about 180 degrees, the “J-shaped” or displaced position. Accordingly, elongated tubular member 20 may be translated to the displaced position, as shown in FIG. 6, for viewing a defect “D₂” positioned on an internal surface of tissue “T” at an angle of about 180 degrees with respect to longitudinal axis “A.” Alternatively, elongated tubular member 20 may be fixed between the substantially overlapping, or linear position (FIG. 5) and the displaced or “J-shaped” curved position (FIG. 6) via the locking mechanism (not shown) to permit visualization of the joint space at angles between zero degrees and 180 degrees off of longitudinal axis “A.”

Thus, as can be appreciated translating elongated tubular member 20 with respect to outer shaft 30 between the substantially overlapping and displaced positions allows for viewing the joint space between zero degrees and 180 degrees off the longitudinal axis “A” in a first plane. Further, as mentioned above, elongated tubular member 20 may be rotated with respect to outer shaft 30 about longitudinal axis “A” for 360 degree positioning of distal end 22 of elongated tubular member 20 radially with respect to longitudinal axis “A” in a second plane.

Accordingly, translating and/or rotating elongated tubular member 20 with respect to outer shaft 30 and longitudinal axis “A” allows for substantially complete 360 degree visualization of the joint space. This 360 degree view is provided without the distortion common to wide angle lenses. Further, during translation and/or rotation of elongated tubular member 20, outer shaft 30 remains in a fixed position with respect to tissue “T,” thus reducing the likelihood of tissue damage.

When the procedure is complete, but prior to removal of arthroscope 10 from the joint space, elongated tubular member 20 is translated proximally with respect to outer shaft 30 to move elongated tubular member 20 to the substantially overlapping position, wherein elongated tubular member 20 is deformed to the shape of outer shaft 30 and positioned within lumen 32 of outer shaft 30. Then, arthroscope 10, defining a minimum diameter and a substantially linear configuration, may be removed from the opening in tissue “T.”

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A surgical instrument comprising:
an elongated tubular member having a proximal end and a distal end, at least a portion of the elongated tubular member being resiliently deformable from an at-rest configuration;
a camera assembly disposed within the elongated tubular member, the camera assembly configured to transmit an image from the distal end of the elongated tubular member to a remotely positioned video display for displaying the image as a video image; and
an outer shaft defining a lumen extending therethrough, the outer shaft disposed annularly about the elongated tubular member, at least one of the outer shaft and the elongated tubular member being moveable with respect to the other between a substantially overlapping position wherein the elongated tubular member is deformed to the shape of the outer shaft, and a displaced position wherein at least a portion of the elongated tubular member extends distally from a distal end of the outer shaft such that the portion of the elongated tubular member extending from the outer shaft returns to the at-rest configuration.

2. The surgical instrument according to claim 1, wherein the elongated tubular member is formed from a shape memory material.

3. The surgical instrument according to claim 1, wherein the elongated tubular member is formed from nitinol.

4. The surgical instrument according to claim 1, wherein the outer shaft defines a substantially linear configuration.

5. The surgical instrument according to claim 1, wherein the distal end of the elongated tubular member is curved off a longitudinal axis of the elongated tubular member by about zero degrees to about 180 degrees with respect to the longitudinal axis of the elongated tubular member when the elongated tubular member is in the at-rest configuration.

6. The surgical instrument according to claim 1, wherein the image is an image of an area extending outwardly from and in the direction of the distal end of the elongated tubular member.

7. The surgical instrument according to claim 1, wherein the elongated tubular member is rotatable with respect to the outer shaft about a longitudinal axis of the outer shaft.

8. The surgical instrument according to claim 1, wherein the outer shaft is formed from one of a rigid and a semi-rigid material.

9. A surgical camera comprising:
an elongated tubular member having a proximal end and a distal end, the elongated tubular member being resiliently deformable between an at-rest curved configuration and a substantially linear configuration;
an image sensing and transmitting assembly disposed within the elongated tubular member, the image sensing and transmitting assembly configured to receive an optical image at the distal end of the elongated tubular and to transmit the image through the elongated tubular member, the image sensing and transmitting assembly adapted to communicate the image to a remotely positioned video display for displaying the image as a video image; and
an outer shaft disposed annularly about the elongated tubular member, at least one of the outer shaft and the elongated tubular member being longitudinally slidable with respect to the other between a substantially overlapping position wherein the elongated tubular member is in the substantially linear configuration and a displaced position wherein the elongated tubular member is returns to the at-rest curved configuration.

10. The surgical camera according to claim 9, wherein the image is an image of an area extending distally along a longitudinal axis of the outer shaft when the elongated tubular member is in the substantially linear configuration and wherein the image is an image of an area off the longitudinal axis of the elongated tubular member, extending from and in the direction of the distal end of the elongated tubular member when the elongated tubular member is in the at-rest curved configuration.

11. The surgical camera according to claim 9, wherein the elongated tubular member is formed from a shape memory material.

12. The surgical camera according to claim 9, wherein the elongated tubular member is formed from nitinol.

13. The surgical camera according to claim 9, wherein the distal end of the elongated tubular member is curved from the substantially linear configuration by about zero degrees to about 180 degrees when the elongated tubular member is in the at-rest curved configuration.

14. The surgical camera according to claim 9, wherein the elongated tubular member is rotatable with respect to the outer shaft about a longitudinal axis of the outer shaft.

15. The surgical camera according to claim 9, wherein the outer shaft is formed from one of a rigid and a semi-rigid material.
